(19) United States
(12) Patent Application Publication
(10) Pub. No.: US 2014/0025085 A1
(43) Pub. Date: Jan. 23, 2014

(54) CATHETER HAVING RADIALLY EXPANDABLE SHAFT

(71) Applicant: BOSTON SCIENTIFIC SCIMED, INC., Maple Grove, MN (US)

(72) Inventors: Pu Zhou, Maple Grove, MN (US); Huisun Wang, Maple Grove, MN (US); James Q. Feng, Maple Grove, MN (US); Dongming Hou, Plymouth, MN (US)

(21) Appl. No.: 13/893,399

(22) Filed: May 14, 2013

Related U.S. Application Data
(60) Provisional application No. 61/672,946, filed on Jul. 18, 2012.

(51) Int. Cl. A61M 25/10; A61B 17/12; A61B 17/42

(52) U.S. Cl. CPC A61M 25/1002; A61B 17/42; A61B 17/12159; A61M 25/1029; A61B 17/12109
USPC 606/119; 156/149; 427/2.3

(57) ABSTRACT

A catheter is disclosed, which is capable of delivering embolization beads for treating uterine fibroids. The elongate catheter shaft includes a radially expandable portion at its distal end, which can expand radially locally when an embolization bead passes through it. In some cases, the catheter shaft includes a non-expandable portion, with an inner diameter comparable to the bead diameter, between the handle and the radially expandable portion. In some cases, the radially expandable portion is made from a tubular braid, stretched longitudinally over a mandrel, and encased in a lubricious soft polymer while still stretched. In other cases, the radially expandable portion is made from a slotted nitinol tube, encased in a lubricious soft polymer below the nitinol transition temperature. The polymer is thick enough to prevent the encased element from returning to its unstretched diameter or a larger size above the transition temperature.

### RELATED APPLICATIONS

This application claims the benefit of U.S. Provisional Application No. 61/672,946 filed Jul. 18, 2012.

### TECHNICAL FIELD

The present disclosure relates to a catheter or microcatheter for treating uterine fibroids.

### BACKGROUND

Uterine fibroids are benign tumors that develop in the uterus. A common treatment for uterine fibroids is uterine artery embolization. In this treatment, a relatively small catheter (sometimes called a microcatheter) is inserted into the bloodstream of the patient and positioned near the fibroids. Small particles or beads are delivered through the catheter and are deposited in the uterine arteries. The beads block or limit the blood supply to the fibroids, which may shrink the fibroids and prevent future growth.

There are two main issues with the microcatheters that are currently used to treat uterine artery embolization.

First, because the outer diameter of the beads is often larger than the inner diameter of the lumen of the catheter, a high pressure is required to force the beads through the lumen. In many cases, this high pressure is supplied manually by the practitioner's hand, through a syringe or plunger on the device handle. Supplying such a high pressure may be problematic for some practitioners.

Second, because the small beads are forced through an even smaller lumen, some beads may deform beyond their yield strain, and may emerge from the catheter with a permanent, oval-shaped deformation. It is desirable that the beads maintain their original generally round shape after being deposited, so such a deformation is undesirable.

Accordingly, there exists a need for a microcatheter that can deliver embolization beads without using excessively high pressure, and without significantly deforming the beads.

### SUMMARY

An embodiment is a catheter. The catheter includes a handle at a proximal end of the catheter. The catheter includes a loading port disposed on the handle. The loading port is capable of receiving an embolization bead. The catheter includes an elongate shaft extending distally from the handle. The catheter includes a radially expandable portion on the elongate shaft. The radially expandable portion has an inner diameter smaller than a diameter of the embolization bead. The embolization bead is capable of being forced distally through the elongate shaft by pressure applied from the handle. As the embolization bead travels distally through the radially expandable portion, the radially expandable portion expands radially locally in the vicinity of the embolization bead.

Another embodiment is a method of forming a radially expandable portion of an elongate catheter shaft. A tubular braid is formed. The braid is longitudinally stretched over a mandrel to produce a longitudinally stretched braid having a smaller diameter than the tubular braid. The longitudinally stretched braid is encased in a lubricious soft polymer. The lubricious soft polymer is sufficiently thick to prevent the longitudinally stretched braid from returning to the size of the tubular braid. The mandrel is removed.

Another embodiment is a method of forming a radially expandable portion of an elongate catheter shaft. A nitinol tube is provided. The nitinol tube has a transition temperature between room temperature and human body temperature. At a temperature below the transition temperature, a plurality of slots is formed in the nitinol tube to produce a slotted tube. At a temperature below the transition temperature, the slotted tube is disposed over a mandrel. At a temperature below the transition temperature, the slotted tube is encased in a lubricious soft polymer. At a temperature below the transition temperature, the mandrel is removed.

### BRIEF DESCRIPTION OF THE DRAWINGS

The foregoing and other objects, features and advantages disclosed herein will be apparent from the following description of particular embodiments disclosed herein, as illustrated in the accompanying drawings in which like reference characters refer to the same parts throughout the different views. The drawings are not necessarily to scale, emphasis instead being placed upon illustrating the principles disclosed herein.

FIG. 1 is side-view drawing of an example catheter and embolization bead.

FIGS. 2A-2D are side-view drawings of an embolization bead progressing distally through the distal end of the catheter shaft.

FIG. 3 is side-view drawing of another example catheter and embolization bead.

FIGS. 4A-4E are side-view drawings of an embolization bead progressing distally through the distal end of the catheter shaft.

FIGS. 5A-5D are side-view drawings of a braided tube being formed into a radially expandable portion of the catheter shaft.

FIGS. 6A-6E are side-view drawings of a slotted tube being formed into a radially expandable portion of the catheter shaft.

### DETAILED DESCRIPTION

In this document, for all of the following descriptions, “proximal” is intended to mean the end closest to the practitioner, “distal” is intended to mean the end farthest away from the practitioner, and “longitudinal” is intended to mean extending between the proximal and distal ends.

A catheter is disclosed, which is capable of delivering embolization beads for treating uterine fibroids. The elongate catheter shaft includes a radially expandable portion at its distal end, which can expand radially locally when an embolization bead passes through it. In some cases, the catheter shaft includes a non-expandable portion, with an inner diameter comparable to the bead diameter, between the handle and the radially expandable portion. In some cases, the radially expandable portion is made from a tubular braid, stretched longitudinally over a mandrel, and encased in a lubricious soft polymer while still stretched. In other cases, the radially expandable portion is made from a slotted nitinol tube, encased in a lubricious soft polymer below the nitinol transition temperature. The polymer is thick enough to prevent the encased element from returning to its unstretched diameter or a larger size above the transition temperature.

The above paragraph is merely a generalization of several of the elements and features described in detail below, and should not be construed as limiting in any way.

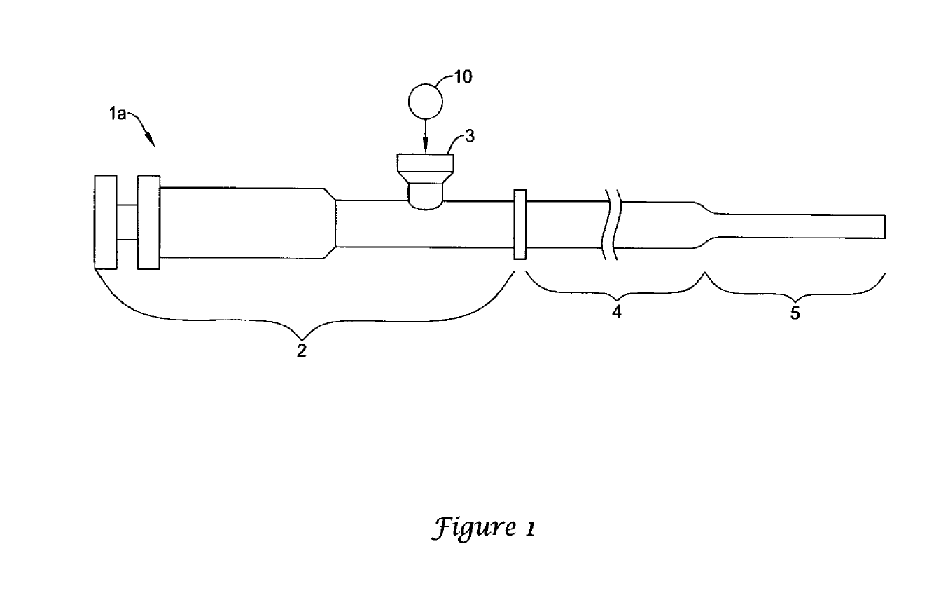

FIG. 1 is side-view drawing of an example catheter 1a and embolization bead 10.

The embolization beads 10 are typically small spheres, and are sometimes referred to as microbeads. The beads 10 are commercially available in various diameters, typically from about 0.5 mm to about 1 mm, and various materials, such as acrylic copolymers and PDA. Some beads 10 may be formed as layered structures, which may include more than one material. Depending of materials, structure and size, some beads 10 may be more elastic than others. It is envisioned that the catheters described herein will work with many, if not all, known beads 10, regardless of the elasticity of the beads 10, which is advantageous.

The catheter 1a of FIG. 1, which does not include the bead 10, has a handle 2 at its proximal end. The handle 2 is generally hand-held and is manipulated by the practitioner during an embolization procedure. The handle may include a thumb-depressible or a finger-depressible syringe or plunger that supplies the pressure for forcing the bead 10 distally through the shaft. Such a syringe may have a button on the proximal end of the handle, as drawn in FIG. 1. Alternatively, the syringe may be activated by a finger-pulled trigger, or any other activation device.

The handle 2 may include a loading port 3, which is capable of receiving an embolization bead 10. The loading port 3 is drawn as a simple opening in FIG. 1, but due to the small size of the bead 10, it is understood that the loading port 3 may include additional structure to aid the practitioner in handling such small objects. In general, ports that load objects into catheters are known.

The catheter 1a includes an elongate shaft extending distally from the handle 2. Once a bead 10 is loaded into the catheter through the loading port 3 on the handle 2, it is advanced distally down the elongate shaft by pressure supplied by the practitioner pressing on the syringe. It is assumed that the handle 2 and the shaft are mated in a known manner, with a suitable connection that allows the bead 10 to pass easily from the handle 2 to the shaft.

The catheter shaft of FIG. 1 includes a proximal portion 4 directly adjacent to the handle 2. This proximal portion 4 has a relatively large inner diameter, typically comparable to the diameter of the embolization bead 10, so that as the embolization bead 10 travels distally through the proximal portion 4, there is no need for the proximal portion 4 to expand radially locally in the vicinity of the embolization bead 10.

There may be advantages to using the relatively large-inner-diameter proximal portion 4. First, the distal travel of the bead 10 may be initiated with relatively little pressure from the handle. Second, there is no deformation of the bead 10 in the proximal portion 4 of the shaft. Third, the relatively large size may improve navigation within large blood vessels. More specifically, the relatively large proximal portion 4 may be easier to push forward in the large vessel that make up most of the path to the target site, when compared with a typical known catheter.

The relatively large inner diameter cannot be used all the way to the distal end of the shaft, though, because it would be too large for the vessels close to the target site. Instead, the shaft includes a radially expandable portion 5 at its distal end, which has an inner diameter smaller than both the inner diameter of the proximal portion 4 and the diameter of the bead 10. Such a radially expandable portion 5 may be relatively short in length, compared with the relatively long proximal portion 4, since the small diameter is only required relatively close to the target site.

The radially expandable portion 5 is intended to expand locally as a bead 10 is passed through it. Typically, the expansion is piece-wise down the length of the radially expandable portion 5, with the expansion occurring only in the immediate vicinity of the bead 10, and the expansion following the bead 10 distally from the proximal portion 4 to its exit at the distal end of the radially expandable portion 5. Alternatively, it is also possible for the full radially expandable portion 5 to expand all at once, and remain expanded during the entire travel of the bead 10 through it.

Some typical diameters for the catheter shaft portions are as follows. A typical inner diameter for the proximal portion 4 is 0.055 inches (1.40 mm) to 0.058 inches (1.47 mm). A typical outer diameter for the proximal portion is 0.065 inches (1.65 mm), which corresponds to a catheter size of 5F. A typical inner diameter for the radially expandable portion 5 is 0.020 inches (0.5 mm) to 0.027 inches (0.7 mm). A typical outer diameter for the radially expandable portion 5 is 0.030 inches (0.77 mm) or 0.035 inches (0.90 mm), which corresponds to a catheter size of 2.3F or 2.7F. It is understood that any suitable size may be used, and not just the sizes listed here.

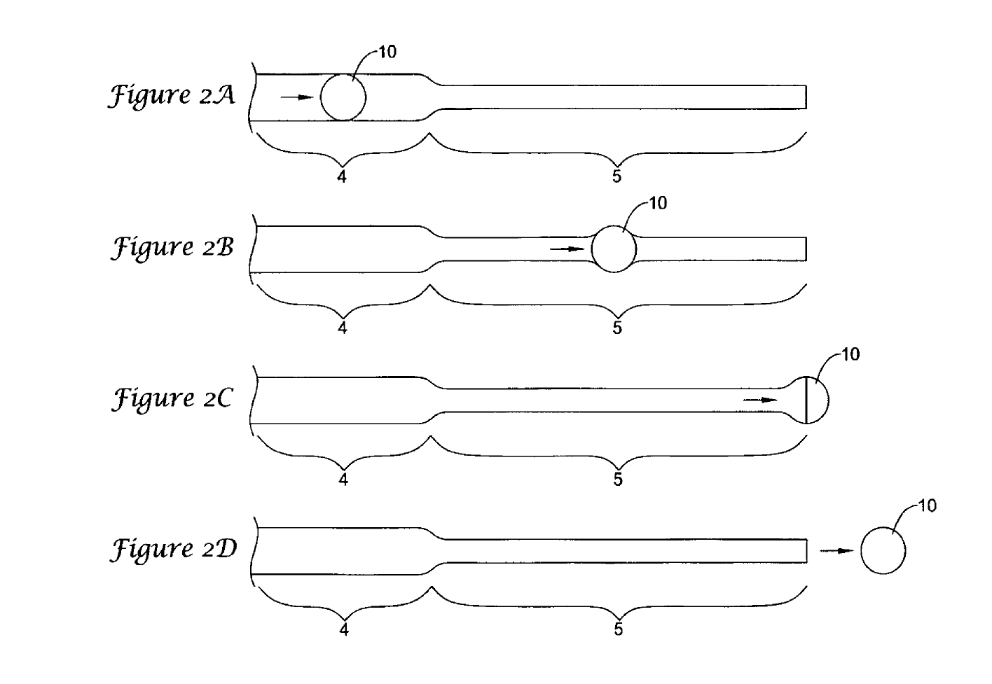

FIGS. 2A-2D are side-view drawings of an embolization bead 10 progressing distally through the distal end of the catheter shaft. In FIG. 2A, the bead 10 is still in the proximal portion 4. In FIG. 2B, the bead 10 has entered the radially expandable portion 5. Note the local radial expansion right around the bead 10, which tapers off on either longitudinal side of the bead 10. In FIG. 2C, the bead 10 is just emerging from the distal end of the radially expandable portion 5. In FIG. 2D, the bead 10 has exited the radially expandable portion 5. Here, the bead 10 is still round, and has not been deformed by passage through the shaft. Plus, the radially expandable portion 5 has returned to its original, un-expanded size throughout.

There may be advantages to using the relatively short radially expandable portion 5 at the distal end of the shaft. First, the relatively small outer diameter improved maneuverability inside the small vessels near the target site. Second, the catheter lumen may inflate to a larger inner diameter under the injection pressure. This, in turn, may reduce deformation of the bead 10 and may create less pressure against the catheter lumen wall when the bead 10 passes through the catheter lumen. The reduction of pressure may result in less friction force between the bead 10 and the lumen wall, and may therefore require less injection pressure to sustain the movement of the bead 10. Third, the radially expandable portion 5 may be made of or made with a highly elastic polymer. Once the pressure is removed, the radially expandable portion 5 may recover back to its original smaller inner diameter on its own. Fourth, the radially expandable portion 5 may allow for use of harder and less elastic beads than typical known catheter shafts.

Thus far, one configuration has been described, where the elongate shaft has a relatively large-diameter proximal portion adjacent or directly adjacent to the handle, followed by a relatively small inner-diameter distal portion, where the distal portion 2 is radially expandable as a bead 10 passes through it. For this configuration, the proximal portion 4 is generally longer than the radially expandable portion 5, since the relatively small-diameter portion is required only near the target site. Alternatively, the proximal portion 4 may be the same length as the radially expandable portion 5 or shorter than the radially expandable portion 5, although these two cases are less common.

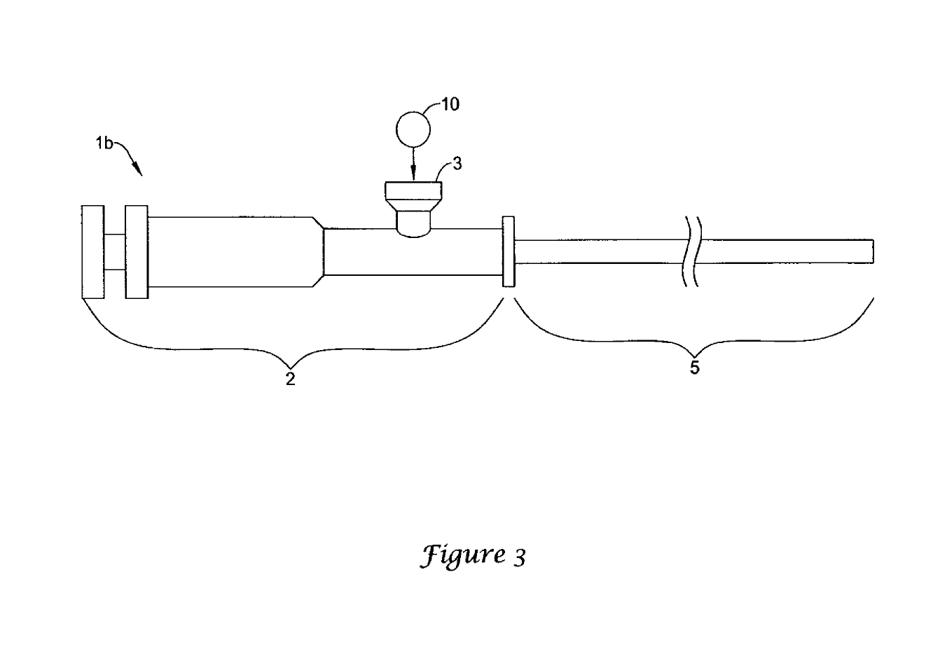

FIG. 3 is side-view drawing of another configuration, for the catheter 1b and embolization bead 10. In this configuration, there is no relatively large-inner-diameter proximal portion, and the radially expandable portion 5 extends distally from the handle 2 (or extends from an adapter at or near the handle, not shown), out to a distal end. Here, the elongate shaft may simply be the radially expandable portion 5 along its entire length.

Many of the advantages of the configuration of FIG. 1 are present in the configuration of FIG. 3, including the use of a reduced injection pressure to move the bead 10 through the catheter shaft, and a reduction or elimination of deformation to the bead 10 itself.

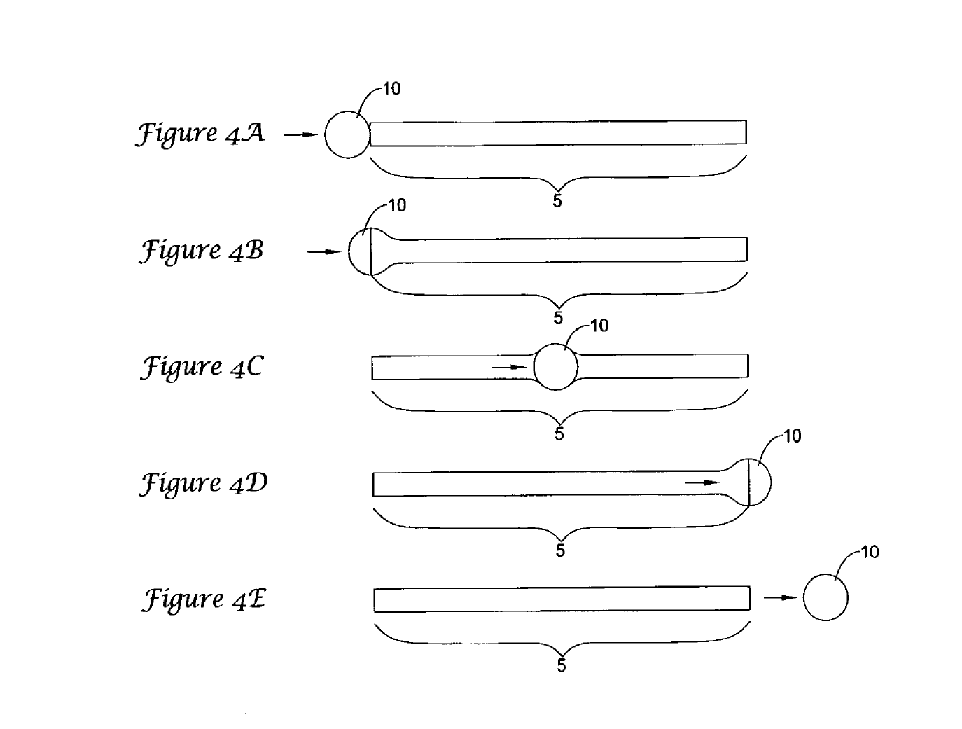

FIGS. 4A-4E are side-view drawings of an embolization bead 10 progressing distally through the distal end of the catheter shaft. More specifically, FIGS. 4A-4E show the bead 10 progressing distally through the radially expandable portion 5, analogous to FIGS. 2A-2D.

There are various structures that may be used for the radially expandable portion 5. A first example is a braided tube 6a, which may be stretched longitudinally and encased in a lubricious soft polymer 9, which can be commercially available and sold under names as such as Tecophilic 83A. A second example is a slotted nitinol tube 6b, which may also be encased in a lubricious soft polymer 9. Both of these examples are described more fully below.

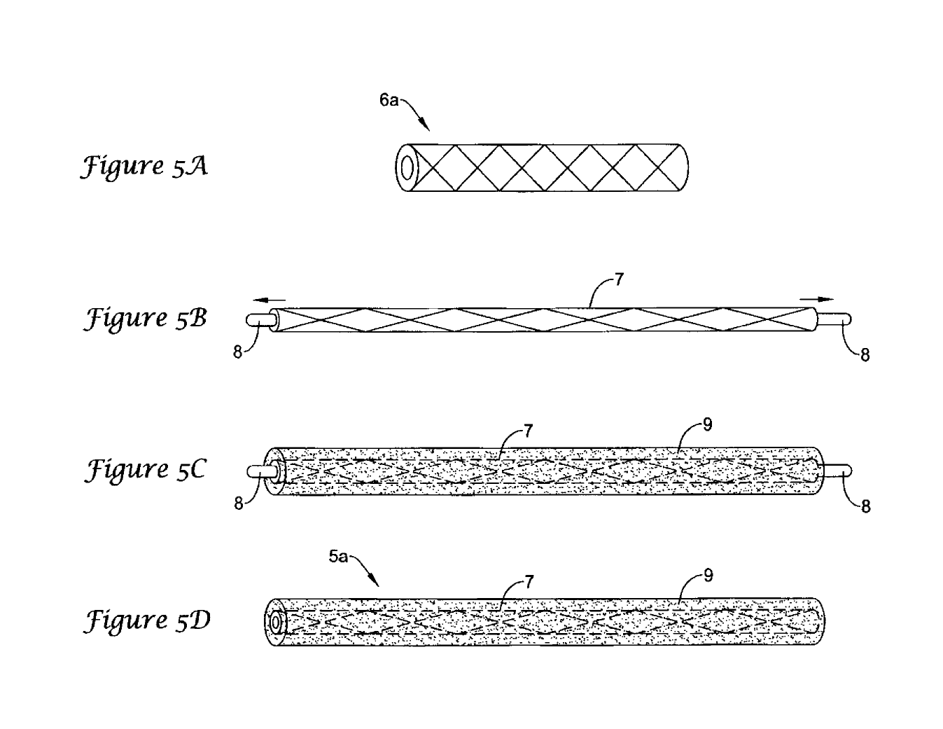

FIGS. 5A-5D are side-view drawings of a braided tube 6a being formed into a radially expandable portion 5a of the catheter shaft.

In FIG. 5A, a formed tubular braid 6a is shown. The braid 6a is made with a relatively large inner diameter, such as the inner diameter used with a 5F catheter size. The braid 6a may have any suitable specific internal structure, such as one-over one, one-over two, two-over-two, and so forth. The wires of the braid 6a may have any suitable shape, with cross-sections that may be round, square, triangle, ribbon-shaped and so forth. For simplicity, the wire or wires of the braid 6a in FIG. 5A are drawn simply as cross-hatching, although it will be understood that any suitable wires and wire configurations may be used. In addition, the wires may be formed from any suitable material, such as one or a combination of metals, polymers, blends and/or alloys.

In FIG. 5B, the braid 6a is longitudinally stretched over a mandrel 8. This produces a longitudinally stretched braid 7 having a smaller diameter than the tubular braid 6a. The mandrel 8 may be generally cylindrical and elongated in shape, may be coated with a material such as PTFE, and may be sized appropriately, such as with an outer diameter of 0.027 inches (0.7 mm). Other suitable sizes, shapes and materials may be used as well. Although not shown in FIG. 5B, the braid may be fixedly attached or tied to the mandrel 8 at both ends so that it cannot spring back to its original length and original diameter.

In FIG. 5C, the longitudinally stretched braid 7 is encased in a lubricious soft polymer 9. In some cases. The polymer 9 is reflowed over the longitudinally stretched braid 7.

The thickness of the polymer 9 depends on the precise geometry and materials for the braid, but in general, the polymer should be thick enough so that when released from the mandrel 8, the polymer 9 prevents the braid 7 from returning to its original unstretched size, namely that of the tubular braid 6a. If the polymer 9 is made too thick, it will stiffen the longitudinally stretched braid 7 and will reduce the amount that it can radially expand, under the influence of a given inflation pressure. As such, in general, the radially expandable portion 5a has a radial expandability that depends on the thickness of the lubricious soft polymer 9; the thinner the polymer wall, the easier it is to inflate the tube.

Finally, in FIG. 5D, the mandrel 8 is removed, leaving the completed radially expandable portion 5a. If the braid 7 is previously attached or tied to the mandrel 8, it is detached or untied before the mandrel 8 is removed.

Note that in the radially expandable portion 5a of FIG. 5D, formed from the tubular braid 6a, the lubricious soft polymer 9 exerts a restraining force on the braid 7 to keep it in its longitudinally stretched state. Without such a restraining force, the braid 7 would return to its original unstretched size and shape, as in FIG. 5A.

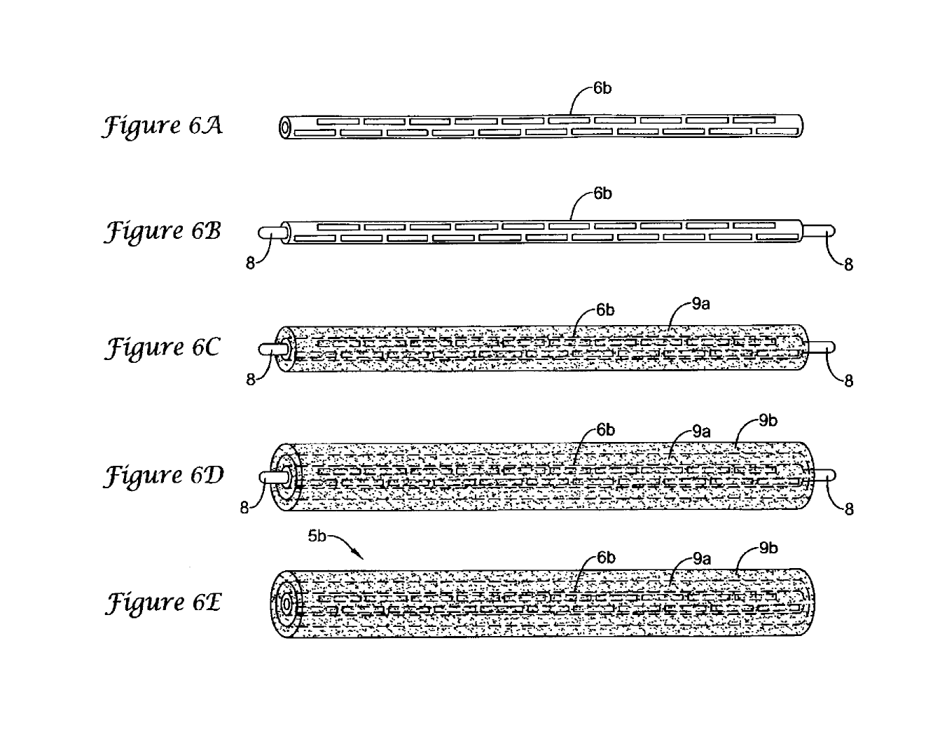

FIGS. 6A-6E are side-view drawings of a slotted tube 6b being formed into a radially expandable portion 5b of the catheter shaft.

First, a tube is provided, made of nitinol, although any suitable shape memory material may also be used. The nitinol tube may have a transition temperature between room temperature and human body temperature. Below the transition temperature, the nitinol may have a relatively small size. Above the transition temperature, the nitinol may have a relatively large size. To form the radially expandable portion 5b shown and described herein, the steps below are all performed below the transition temperature, such as at room temperature or some other cooled temperature.

Next, slots or holes in the tube are cut, etched or ablated, to form a slotted tube 6b, as shown in FIG. 6A. In FIG. 6A, the slots are oriented generally longitudinally, and are arranged in rows where each row is offset from the adjacent rows. The slots may have any suitable size, shape, orientation and arrangement, as is well known to one of ordinary skill in the art. In some cases, the tube 6b may be cut to resemble a stent.

Note that if the slotted tube 6b were heated above the transition temperature at this stage, it would expand to a larger size. An example of a low-temperature (below the transition temperature, such as room temperature) inner diameter is 0.027 inches (0.7 mm), and of a high-temperature (above the transition temperature, such as human body temperature) inner diameter is 0.055 inches (1.4 mm). These sizes are just examples, and any suitable sizes may be used.

In FIG. 6B, the slotted tube 6b is disposed over a mandrel 8. The mandrel may have a diameter of 0.027 inches (0.7 mm), or any other suitable size. In many cases, the diameter of the mandrel 8 may be matched to the inner diameter to the slotted tube 6b at the relatively low temperatures below the transition temperature. Although not shown in FIG. 6B, the slotted tube 6b may be fixedly attached or tied to the mandrel 8 at both ends so that it cannot significantly change size when it is heated beyond its transition temperature.

In FIG. 6C, the slotted tube 6b, disposed over the mandrel 8, is encased in a layer 9a of the lubricious soft polymer 9. As with the designs of FIGS. 5A-5D described above, it is intended that the lubricious soft polymer 9 exert a restraining force on the element that it surrounds, in order to prevent the element from expanding. For the slotted tube 6b, we want the lubricious soft polymer 9 to be sufficiently thick to prevent the encased slotted tube 6b from substantially changing size as the temperature crosses the transition temperature.

One way to deposit the lubricious soft polymer 9 onto the slotted tube 6b is to dip the slotted tube 6b into a solution of hydrophilic polymer, done at a temperature below the transition temperature. Such a dipping may produce a single layer, as shown in FIG. 6C.

In some cases, it may be that a single layer doesn't have enough thickness to adequately restrain the nitinol tube at higher temperatures. For these cases, the tube 6b may be dipped again. FIG. 6D shows a second layer 9b of the lubricious soft polymer 9, deposited on the first layer 9a of the lubricious soft polymer 9. More layers may be added as needed, although two or three layers are typically sufficient to give enough thickness to the lubricious soft polymer 9.

Finally, FIG. 6E shows the mandrel 8 removed, leaving a completed radially expandable portion 5b.

In some cases, the polymer 9 may be formed as discrete longitudinal segments, along the length of the radially expandable portion 5, 5a, 5b. For instance, the polymer 9 may be formed as a single segment, and then cut into segments afterwards. In other cases, the polymer 9 may be formed in discrete pieces. In general, it is easiest to manufacture elements having five or fewer polymer segments.

It will be understood that the catheters 1a, 1b described herein may also be used for therapies other than for treating uterine fibroids.

It will also be understood that polymers having a variety of elasticities may be used, as long as one adjusts the thickness of the polymers appropriately. In general, the polymer coating of an element should be thick enough to prevent the element from expanding in diameter of its own volition, whether through resistance to an applied longitudinal expansion or through shape memory.

Unless otherwise stated, use of the words “substantial” and “substantially” may be construed to include a precise relationship, condition, arrangement, orientation, and/or other characteristic, and deviations thereof as understood by one of ordinary skill in the art, to the extent that such deviations do not materially affect the disclosed methods and systems.

Throughout the entirety of the present disclosure, use of the articles “a” or “an” to modify a noun may be understood to be used for convenience and to include one, or more than one, of the modified noun, unless otherwise specifically stated.

Elements, components, modules, and/or parts thereof that are described and/or otherwise portrayed through the figures to communicate with, be associated with, and/or be based on, something else, may be understood to so communicate, be associated with, and or be based on in a direct and/or indirect manner, unless otherwise stipulated herein.

Although the methods and systems have been described relative to a specific embodiment thereof, they are not so limited. Obviously many modifications and variations may become apparent in light of the above teachings. Many additional changes in the details, materials, and arrangement of parts, herein described and illustrated, may be made by those skilled in the art.

What is claimed is:

1. A catheter, comprising:
a handle at a proximal end of the catheter;
a loading port disposed on the handle, the loading port being capable of receiving an embolization bead;
an elongate shaft extending distally from the handle; and
a radially expandable portion on the elongate shaft;
wherein the radially expandable portion has an inner diameter smaller than a diameter of the embolization bead;
wherein the embolization bead is capable of being forced distally through the elongate shaft by pressure applied from the handle; and
wherein as the embolization bead travels distally through the radially expandable portion, the radially expandable portion expands radially locally in the vicinity of the embolization bead.

2. The catheter of claim 1, wherein the radially expandable portion comprises a braided tube, stretched longitudinally, and encased in a lubricious soft polymer that maintains the longitudinally stretched shape of the braided tube.

3. The catheter of claim 1,
wherein the radially expandable portion comprises a slotted nitinol tube having a transition temperature of less than or equal to human body temperature;
wherein the slotted nitinol tube is encased in a lubricious soft polymer at a temperature lower than the transition temperature;
wherein the slotted nitinol tube has an increased radial dimension at a temperature greater than its transition temperature.

4. The catheter of claim 1,
wherein the elongate shaft includes a proximal portion between the handle and the radially expandable portion;
wherein the proximal portion has an inner diameter comparable to a diameter of the embolization bead; and
wherein as the embolization bead travels distally through the proximal portion, the proximal portion does not expand radially locally in the vicinity of the embolization bead.

5. The catheter of claim 4, wherein the radially expandable portion has a length shorter than that of the proximal portion.

6. The catheter of claim 4, wherein the radially expandable portion has an inner diameter less than or equal to half that of the proximal portion.

7. The catheter of claim 1, wherein the elongate shaft comprises the radially expandable portion along its entire length.

8. The catheter of claim 1, wherein the embolization bead emerges from a distal end of the radially expandable portion having the generally the same size and shape as before it enters the loading port on the handle.

9. The catheter of claim 1, wherein after the embolization bead emerges from a distal end of the radially expandable portion, the radially expandable portion returns to the same outer diameter as before the embolization bead is received by the loading port on the handle.

10. The catheter of claim 1, wherein the lubricious soft polymer is elastic.

11. The catheter of claim 1, wherein the handle includes a thumb- or finger-depressible syringe that supplies the pressure for forcing the embolization bead distally through the elongate shaft.

12. A method of forming a radially expandable portion of an elongate catheter shaft, comprising:
forming a tubular braid;
longitudinally stretching the braid over a mandrel to produce a longitudinally stretched braid having a smaller diameter than the tubular braid;
encasing the longitudinally stretched braid in a lubricious soft polymer, the lubricious soft polymer being sufficiently thick to prevent the longitudinally stretched braid from returning to the size of the tubular braid; and
removing the mandrel.

13. The method of claim 12, wherein the lubricious soft polymer is reflowed over the longitudinally stretched braid.

14. The method of claim 12, wherein the radially expandable portion has a radial expandability that depends on the thickness of the lubricious soft polymer.

15. The method of claim 12, further comprising:
before the encasing step, fixedly attaching both longitudinal ends of the longitudinally stretched braid to the mandrel; and
between the encasing and removing steps, detaching both longitudinal ends of the longitudinally stretched braid from the mandrel.

16. A method of forming a radially expandable portion of an elongate catheter shaft, comprising:
providing a nitinol tube, the nitinol tube having a transition temperature between room temperature and human body temperature;
at a temperature below the transition temperature, forming a plurality of slots in the nitinol tube to produce a slotted tube;
at a temperature below the transition temperature, disposing the slotted tube over a mandrel;
at a temperature below the transition temperature, encasing the slotted tube in a lubricious soft polymer; and
at a temperature below the transition temperature, removing the mandrel.

17. The method of claim 16, wherein the lubricious soft polymer is sufficiently thick to prevent the encased slotted tube from substantially changing size as the temperature crosses the transition temperature.

18. The method of claim 16, further comprising:
before the encasing step, fixedly attaching both longitudinal ends of the slotted tube to the mandrel; and
between the encasing and removing steps, detaching both longitudinal ends of the slotted tube from the mandrel.

19. The method of claim 16, wherein the encasing step comprises dipping the slotted tube into the lubricious soft polymer to produce a layer of lubricious soft polymer on the slotted tube.

20. The method of claim 16, wherein the encasing step further comprises dipping the slotted tube into the lubricious soft polymer to produce a second layer of lubricious soft polymer on the layer lubricious soft polymer.
